(19) United States
(12) Patent Application Publication
(10) Pub. No.: US 2024/0350383 A1
(43) Pub. Date: Oct. 24, 2024

(54) CLEANSING COMPOSITION

(71) Applicant: L'OREAL, PARIS (FR)

(72) Inventors: Angela ARPINO, BRICK, NJ (US); Hetal Mehul ALATAR, OLD BRIDGE, NJ (US); Paula CZYRIAK, EATONTOWN, NJ (US); Sabina GOSTO DERSH, WESTFIELD, NJ (US); William FEUER, HILLSBOROUGH, NJ (US)

(73) Assignee: L'OREAL, PARIS (FR)

(21) Appl. No.: 18/305,689

(22) Filed: Apr. 24, 2023

(51) Int. Cl. A61K 8/368; A61Q 19/10

(52) U.S. Cl. CPC A61K 8/368; A61Q 19/10; A61K 2800/596

(57) ABSTRACT

The present disclosure relates to cleanser compositions, which generally include a. salicylic acid; b. a first surfactant sodium cocoyl isethionate; c. at least one acrylate copolymer; d. a water insoluble ingredient. The cleanser compositions contain a high level of salicylic acid and exhibits very good cleansing properties as well as being stable.

### FIELD OF THE DISCLOSURE

The present disclosure relates to a cleanser composition. More particularly, the disclosure relates to a cleanser composition comprising a high level of salicylic acid.

### BACKGROUND

Salicylic acid is proven to be effective to address skin conditions that include mild to moderate acne. Products with high concentrations of salicylic acid offer opportunity for addressing acne, however, such compositions can prove challenging. Not only are there practical limits on the amount of salicylic acid that can be included in consumer applied compositions, but there are also limits on formulating cleansers that are shelf stable and offer a pleasing comfortable experience for consumers.

The inventors have overcome these challenges and provide a cleansing composition that remains stable over time at ambient temperatures and resists freeze/thaw cycles wherein the stability is demonstrated in minimal change in pH and viscosity and visible properties of the cleansing composition.

### SUMMARY OF THE DISCLOSURE

The instant disclosure relates to cleanser compositions with high levels of salicylic acid used for acne treatment. These types of cleanser compositions that contain high level of salicylic acid are difficult to stabilize when attempting to suspend water insoluble materials and maintain viscosity. The disclosure provides, in various embodiments, cleanser compositions that contain high levels of salicylic acid and a water insoluble material capable of being suspended and formulated with only bio-based surfactants, free of petroleum derived surfactants such as sulfated or sulfonated types. The viscosity is maintained through stability lifetime.

The instant disclosure relates to cleanser compositions that include:
a. salicylic acid;
b. a first surfactant sodium cocoyl isethionate;
c. at least one one acrylate copolymer;
d. a water insoluble ingredient; and
wherein the cleanser is stable and the water insoluble ingredient stays in suspension; and
wherein all weight percentages are based on a total weight of the cleanser composition.

In some embodiments, the salicylic acid may be present from about 0.5 to 2 wt. % based on the total weight of the cleanser composition.

In various embodiments, the sodium cocoyl isethionate may be present from about 0.5 to 10 wt. % based on the total weight of the cleanser composition.

In some embodiments, the at least one one acrylate copolymer may be present from about 0.1 to 4 wt. % based on the total weight of the cleanser composition.

In some embodiments, the water insoluble ingredient may be present from about 0.05 to about 10 wt. % based on the total weight of the cleanser composition.

In various embodiments, the water insoluble ingredient is selected from the group consisting of hectorite, clay, pearlescent, exfoliants and mixture thereof.

In some embodiments, the cleanser compositions may further include a second or more surfactants. In some embodiments, the second or more surfactants may be selected from the group consisting of anionic, ionic, amphoteric surfactants and mixture thereof.

In some embodiments, the second or more surfactants may be present from about 0.5 to about 20 wt. % based on the total weight of the cleanser composition.

In some embodiments, the composition is directed to a cleanser comprising:
a. from about 0.5 to 2 wt. % of salicylic acid;
b. from about 0.5 to 10 wt. % of a first surfactant sodium cocoyl isethionate;
c. from about 0.1 to 4 wt. % of at least one acrylate copolymer;
d. from about 0.05 to about 10 wt. % of a water insoluble ingredient; and
wherein the cleanser is stable and the water insoluble ingredient stays in suspension; and
wherein all weight percentages are based on a total weight of the cleanser composition.

In some embodiments, the cleanser compositions may further comprise one or more a adjuvants commonly used in the cosmetic field selected from the group consisting of cosmetic or dermatological active principles, fragrances, preservatives, sequestering agents, pigments, pearlescent agents, inorganic or organic fillers, powder or polyethylene powder, soluble dyes, sunscreen agents and their mixtures

The instant disclosure is also directed to methods for cleansing the keratin materials, preferably the skin, more preferably the face, comprising a step of applying the cleanser composition to said keratin materials.

The instant disclosure is also directed to the use of cleanser compositions in cleansing the keratin material, preferably the skin, more preferably the face.

The cleanser compositions of the instant disclosure provide cleansers with high levels of salicylic acid that are difficult to stabilize when attempting to suspend water insoluble materials and maintain viscosity.

Without being bound by theory or mechanism, it is suggested that the ratio between salicylic acid, sodium cocoyl isethionate and one acrylate copolymer help the water insoluble material to stay suspended.

It is to be understood that both the foregoing general description and the following detailed description are exemplary and explanatory only, and are not restrictive of the invention.

### DETAILED DESCRIPTION OF THE DISCLOSURE

The instant disclosure relates to cleanser compositions with high levels of salicylic acid used for acne treatment.

The instant disclosure relates to cleanser compositions that include:
a. salicylic acid;
b. a first surfactant sodium cocoyl isethionate;
c. at least one acrylate copolymer;
d. a water insoluble ingredient; and
wherein the cleanser is stable and the water insoluble ingredient stays in suspension; and
wherein all weight percentages are based on a total weight of the cleanser composition.

As used herein, the term “essentially sulfate free” means that, while it is preferable that no sulfate is present in the compositions of the invention, it is possible to have very small amounts of sulfate in the compositions, provided that these amounts do not materially affect the advantageous properties of the composition. In particular, “essentially sulfate-free anionic surfactant” means that no traditional sulfate-based anionic surfactants are used in the composition and that the surfactant does not contribute sulfate to the composition. Most preferably, the compositions contain no sulfate. To the extent any sulfate is present in the compositions, it is present at an amount of less than about 2.0% by weight, typically less than about 1.5% by weight, typically less than about 1.0% by weight, typically less than about 0.5% by weight, more typically less than about 0.1% by weight, based on the total weight of the composition. To the extent present, the sulfates in such compositions are typically contributed by components other than the anionic surfactant.

### Salicylic Acid

In accordance with the various embodiments, the cleaner composition according to the disclosure comprises salicylic acid.

Salicylic acid (from Latin salix, willow tree) is a lipophilic monohydroxybenzoic acid, a type of phenolic acid, and a beta hydroxy acid (BHA). It has the formula C7H6O3. This colorless crystalline organic acid is widely used in organic synthesis and functions as a plant hormone.

In accordance with the various embodiments, the amount of salicylic acid present in the cleaner composition is in a range from about 0.5 to about 2 wt. %, or from 0.55 to about 1.95 wt. %, or from about 0.6 to about 1.9 wt. %, or from about 0.65 to about 1.85 wt. %, or from about 0.7 to about 1.8 wt. %, or from about 0.75 to about 1.85 wt. %, or from about 0.8 to about 1.8 wt. %, or from about 0.85 wt. % to about 1.75 wt. %, or from about 0.9 to about 1.70 wt. %, or from about 0.95 to about 1.65 wt. % or any suitable combination, sub-combination, range, or sub-range thereof by weight, based on the total weight of the composition.

Thus, any one of or a combination of salicylic acid is present, by weight, based on the total weight of the cleaner composition, from about 0.5, 0.6, 0.7, 0.8, 0.9, 1.0 to about 1.0, 1.1, 1.2, 1.3, 1.4, 1.5, 1.6, 1.7, 1.8, 1.9 or 2 wt. % including increments and ranges therein and there between.

### Surfactants

### Sodium Cocoyl Isethionate (SCI)

The Sodium Cocoyl Isethionate (i.e. SCI) may be present in an amount from about 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 2.0 wt. %, 2.2 wt. %, 2.4 wt. %, 2.6 wt. %, 2.8 wt. %, 3.0 wt. %, 3.5 wt. %, 4.0 wt. %, 4.5 wt. %, 5.0 wt. %, 5.5 wt. % to about 5.5 wt. %, 5.6 wt. %, 5.8 wt. %, 6.0 wt. %, 6.5 wt. %, 7.0 wt. %, 7.5 wt. %, 8.0 wt. %, 8.5 wt. %, 9.0 wt. %, 9.5 wt. %, or 10 wt. % or any suitable combination, sub-combination, range, or sub-range thereof by weight, based on the total weight of the composition.

### Other Surfactants

In some embodiments, the one or more surfactants may be selected from the following classes of surfactants: Carboxylates, Alkyl ether carboxylates, Alkyl sarcosinates, Sulfates, Alkyl ether sulfates, Alkyl sulfates, Alkyl taurates, Sulfonates, Alkyl sulfonates, Alkylbenzene sulfonates, Sulfosuccinate esters, Dialkyl sulfosuccinates, Alkyl sulfosuccinates, Paraffin sulfonates, α-olefin sulfonates, Phosphates, Alkyl phosphates, Alkyl ether phosphates, Polyethers, Fatty acid ethoxylates, fatty alcohol ethoxylates, Alkylphenol ethoxylates, Fatty amide ethoxylates, Fatty amine ethoxylates, Ethoxylated triglycerides, Fatty acid methyl ester ethoxylates, Poloxamers, Sucrose esters, Sorbitan esters, Sorbitan alkyl esters, Alkyl glucosides, Alkyl polyglucosides, Sorbitan alkanoate, Ethoxylated sorbitan alkanoates, Acetylenic glycols, N-alkyl derivatives of simple amino acids, Glycinates, Betaines, Amido betaines, Alkyl betaines, Pyrophosphobetaines, Aminopropionates, Alkyl amphopropionates, Alkyl amphodipropionates, Glutamates, Cocoates, Imidazolines, N-oxides of tertiary amines, Alkyl amphoacetate, Alkyl amphodiacetate, Hydroxysultaines and mixtures thereof.

Surfactants in the composition include a single surfactant or a mixture of surfactants (often surfactant powders or in other easily used forms (liquid)). In one embodiment, the composition includes one or more surfactants that includes anionic, zwitterionic, non-ionic surfactants and combinations thereof.

### Zwitterionic Surfactant

Non-limiting zwitterionic surfactants include, for example, coco-betaine, lauryl betaine, oxyethylenated (10 EO) lauryl betaine, oxyethylenated (10 EO) stearyl betaine, cocamidopropyl betaine, lauramidopropyl betaine and mixtures thereof.

The surfactant in the composition according to the instant disclosure may be one zwitterionic surfactant chosen from betaines, (C8-C20)alkyl betaines, (C8-C20)alkylamido(C1-C6)alkylbetaines, and mixtures thereof. Two or more zwitterionic surfactants may be used in combination. Thus, a single type of zwitterionic surfactant or a combination of different types of zwitterionic surfactants may be used.

Examples of zwitterionic surfactants include surfactants selected from these classes of surfactants: N-alkyl derivatives of simple amino acids, Glycinates, Betaines, Amido betaines, Alkyl betaines, Pyrophosphobetaines, Aminopropionates, Alkyl amphopropionates, Alkyl amphodipropionates, Glutamates, Cocoates, Imidazolines, N-oxides of tertiary amines, Alkyl amphoacetate, Alkyl amphodiacetate, Hydroxysultaines. In selected embodiments, the preferred surfactants are those having alkyl and alkylarye chains in the C10 to C18 range.

Mention may in particular be made, as betaines, of (C8-C20)alkyl betaines, such as, for example, coco betaine, such as the product sold under the name Dehyton AB-30® by the company Cognis, lauryl betaine, such as the product sold under the name Genagen KB® by the company Clariant, oxyethylenated (10 EO) lauryl betaine, such as the product sold under the name Lauryl Ether (10 EO) Betaine® by the company Shin Nihon Rica, or oxyethylenated (10 EO) stearyl betaine, such as the product sold under the name Stearyl Ether (10 EO) Betaine® by the company Shin Nihon Rica, DEHYTON AB 30 by the company BASF.

Mention may be made, among (C8-C20)alkylamido(C1-C6)alkylbetaines and derivatives thereof, for example, of cocamidopropyl betaine, sold under the name Lebon 2000 HG® by the company Sanyo or sold under the name Empigen BB® by the company Albright & Wilson, or lauramidopropyl betaine, sold under the name Rewoteric AMB12P® by the company Witco.

Preferably, the zwitterionic surfactant is chosen from (C8-C20)alkyl betaines, (C8-C20)alkylamido(C1-C6)alkylbetaines, and mixtures thereof, and preferably among coco betaine, lauryl betaine, oxyethylenated (10 EO) lauryl betaine, oxyethylenated (10 EO) stearyl betaine, cocamidopropyl betaine and mixtures thereof, and more preferably is selected from lauryl betaine, coco betaine and mixtures thereof and still more preferably is coco betaine.

### Anionic Surfactants

In some embodiments, one surfactant can be, for example, a compound selected from the group consisting of sodium lauroyl glutamate, sodium lauryl sulfate, disodium lauryl sulfosuccinate, diethylhexyl sodium sulfosuccinate, sodium cocoyl glycinate, potassium cocoyl glycinate, and the mixtures thereof.

In some embodiments, one of the surfactants is disodium lauryl sulfosuccinate.

Examples of anionic surfactants include surfactants selected from these classes of surfactants: Carboxylates, Alkyl ether carboxylates, Alkyl sarcosinates, Sulfates, Alkyl ether sulfates, Alkyl sulfates, Alkyl taurates, Sulfonates, Alkyl sulfonates, Alkylbenzene sulfonates, Sulfosuccinate esters, Dialkyl sulfosuccinates, Alkyl sulfosuccinates, Paraffin sulfonates, α-olefin sulfonates, Isethionates, Phosphates, Alkyl phosphates, Alkyl ether phosphates Alternative surfactants may include or be combined with cleansing surfactants or cleansing agents suitable for use in skin cleansers or on skin cleansing fibrous pads.

### Non Ionic Surfactants

In some embodiments, one of the surfactant is PEG-7 Glyceryl Cocoate.

Useful non ionic surfactants include, but are not limited to, the following surfactant classes: Polyethers, Fatty acid ethoxylates, Fatty alcohol ethoxylates, Alkylphenol ethoxylates, Fatty amide ethoxylates, Fatty amine ethoxylates, Ethoxylated triglycerides, Fatty acid methyl ester ethoxylates, Poloxamers, Sucrose esters, Sorbitan esters, Sorbitan alkyl esters, Alkyl glucosides, Alkyl polyglucosides, Sorbitan alkanoate, Ethoxylated sorbitan alkanoates, Acetylenic glycols, and a mixture thereof.

In alternative embodiments, the surfactant may include suitable nonionic surfactants including alkyl polyglucoside having alkyl groups from C10 to C16.

### Other Surfactants

According to the instant disclosure, these surfactants can be chosen from salts, for example, alkali metal salts such as sodium salts, potassium salts, ammonium salts, calcium salts, amine salts, amino alcohol salts and alkaline-earth metal salts.

Examples of alkaline-earth metal salts include magnesium salts of the following types of compounds: acyl isethionates, acyl glycinates, acyl taurates, acyl amino acids, acyl sarcosinates, sulfosuccinates, sulfonates, and sulfoacetates, the alkyl and acyl groups of all these compounds comprising from 6 to 24 carbon atoms (saturated or unsaturated, linear or branched).

Non-limiting examples of acyl amino acids useful in the compositions of the invention include those having the following formula (I):

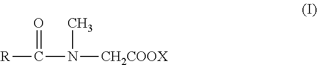

in which
R is a linear or branched, saturated or unsaturated C8-C16 and more preferentially C12-C18 alkyl chain;
X is an organic cation, for instance an alkanolamine such as triethanolamine, or a mineral cation, for instance an alkali metal such as sodium or potassium, or alternatively ammonia.

Among the preferred radicals R, mention may be made of stearyl, myristyl, oleyl, lauryl and cocoyl.

Among the N-acyl sarcosinates that may be used according to the invention, mention may be made of sodium lauroyl sarcosinate (INCI name: Sodium lauroyl sarcosinate) sold under the name Sarkosyl NL 97® by the company Ciba or sold under the name Oramix L 30® by the company SEPPIC, or sold under the name Amin LS30L by the company Guangzhou Tinci Materials; sodium myristoyl sarcosinate (INCI name: Sodium myristoyl sarcosinate) sold under the name Nikkol Sarcosinate MN® by the company Nikkol, sodium palmitoyl sarcosinate (INCI name: Sodium palmitoyl sarcosinate) sold under the name Nikkol Sarcosinate PN® by the company Nikkol.

Use will be made more particularly of sodium N-lauroyl sarcosinate (INCI name: Sodium lauroyl sarcosinate).

Non-limiting examples of taurates useful in the compositions of the invention include those having the following formula (II):

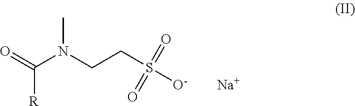

wherein R is selected from H or an alkyl chain that has 1-24 carbon atoms, said chain being saturated or unsaturated, linear or branched.

A particular taurate that can be used in the current compositions is sodium methyl cocoyl taurate.

Non-limiting examples of isethionates useful in the compositions of the invention include those having the formulas (III) or (IV) below:

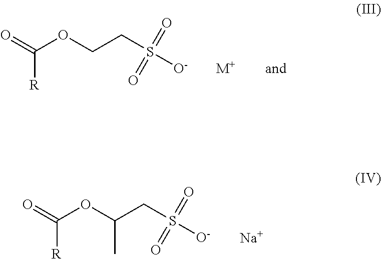

wherein R is selected from H or an alkyl chain that has 1-24 carbon atoms, said chain being saturated or unsaturated, linear or branched.

Particular isethionates that can be used in the current compositions include, for example, sodium cocoyl isethionate, sodium lauroyl methyl isethionate, and mixtures thereof.

Non-limiting examples of sulfosuccinates useful in the compositions of the invention include those having the following formula (V):

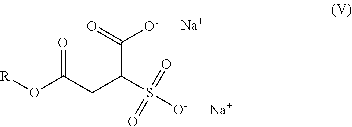

wherein R is selected from H or an alkyl chain that has 1-24 carbon atoms, said chain being saturated or unsaturated, linear or branched.

A particular sulfosuccinate that can be used in the current compositions is disodium laureth sulfosuccinate.

Non-limiting examples of sulfonates useful in the compositions of the invention include those having the following formula (VI):

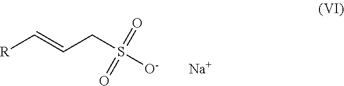

wherein R is selected from H or an alkyl chain that has 1-24 carbon atoms, said chain being saturated or unsaturated, linear or branched.

A particular sulfonate that can be used in the current compositions is sodium C14-16 olefin sulfonate.

Non-limiting examples of sulfoacetates useful in the compositions of the invention include those having the following formula (VII):

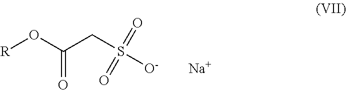

wherein R is as defined above for the sulfonates.

A particular sulfoacetate that can be used in the current compositions is sodium lauryl sulfoacetate.

According to one embodiment, the anionic surfactant that is most preferred in the instant disclosure is acyl amino acids useful in the compositions of the invention include those having the following formula (I) as described above, more preferably N-acyl sarcosinates, even more preferably sodium lauroyl sarcosinate (INCI name: Sodium lauroyl sarcosinate).

### Acrylates Copolymer

The composition of the instant disclosure comprises at least one acrylates copolymer.

Preferably, the acrylates copolymer is selected from crosslinked copolymers containing at least one monomer selected from methacrylic acid; acrylic acid; amino acrylic acid; an acrylic acid ester of a C8-C30 alkyl or alkenyl, which may be substituted; a methacrylic acid ester of a C8-C30 alkyl or alkenyl, which may be substituted; a C1-C4 alkyl acrylate and a C1-C4 methacrylate, which may be substituted.

Particular mention may be made of crosslinked copolymers comprising at least one methacrylic acid unit and at least one C1-C4 alkyl acrylate unit. The crosslinked copolymer comprising at least one methacrylic acid unit and at least one C1-C4 alkyl acrylate unit means a crossklinked copolymer comprised of at least one methacrylic acid unit and at least one alkyl acrylate unit, wherein the alkyl acrylate is chosen from C1-C4 alkyl acrylates.

In the crosslinked copolymer disclosed herein, the at least one methacrylic acid unit can be present in an amount ranging from 20% to 80% by weight, for example, from 25% to 70% by weight, and further, for example, from 35% to 60% by weight, relative to the total weight of the copolymer.

In the crosslinked copolymer disclosed herein, the at least one alkyl acrylate unit can be present in an amount ranging from 15% to 80% by weight, for example, from 25% to 75% by weight, and further, for example, from 40% to 65% by weight, relative to the total weight of the copolymer. The alkyl acrylate can, for example, be chosen from methyl acrylate, ethyl acrylate, propyl acrylate and butyl acrylate. In one embodiment, the alkyl acrylate is ethyl acrylate.

The copolymer disclosed herein is partially or totally crosslinked with at least one crosslinking agent. The at least one crosslinking agent can be chosen, for example, from polyunsaturated compounds, such as polyethylenically unsaturated compounds. These compounds can be chosen, for example, from polyalkenyl ethers of sucrose, polyalkenyl ethers of polyols, diallyl phthalates, divinylbenzene, allyl(meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, castor oil derivatives and polyol derivatives manufactured from unsaturated carboxylic acids.

The at least one crosslinking agent that may also be used include, for example, unsaturated monomers comprising at least one reactive group capable of reacting with an unsaturation to form a crosslinked copolymer.

The content of the at least one crosslinking agent generally ranges from 0.01% to 5% by weight, for example, from 0.03% to 3% by weight, and further, for example, from 0.05% to 1% by weight, relative to the total weight of the copolymer.

According to one embodiment, the at least one crosslinked copolymer disclosed herein may, for example, be in the form of a dispersion in water. The number-average size of the copolymer particles in the dispersion generally ranges from 10 nm to 500 nm, for example, from 20 nm to 200 nm, and further, for example, from 50 nm to 150 nm.

These copolymers are described, for example, in Patent Application No. WO 01/76552.

For example, the crosslinked methacrylic acid/ethyl acrylate copolymer in the form of an aqueous dispersion of 30% active material manufactured and sold under the name Carbopol® Aqua SF-1 by the company LUBRIZOL may be used.

Advantageously, the at least one acrylates copolymer is present in the composition according to the instant disclosure in an amount ranging from about 0.1 to about 4 wt. %, or from about 0.15 to about 3.95 wt. %, or from about 0.2 to about 3.9 wt. %, or from about 0.25 to about 3.85 wt. %, or from about 0.3 to about 3.8 wt. %, or from about 0.35 to about 3.75 wt. %, or from about 0.4 to about 3.7 wt. %, or from about 0.45 to about 3.65 wt. %, or from about 0.5 to about 3.6 wt. %, or from about 0.55 wt. % to about 3.55 wt. %, or from about 0.6 to about 3.5 wt. %, or from about 0.65 to about 3.45 wt. %, or from about 0.7 to about 3.4 wt. %, or from about 0.75 to about 3.35 wt. % or any suitable combination, sub-combination, range, or sub-range thereof by weight, based on the total weight of the composition.

Thus, any one of or a combination of acrylate copolymer is present, by weight, based on the total weight of the cleanser composition, from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 to about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4 wt. % including increments and ranges therein and there between.

### Water Insoluble Ingredient

The composition of the instant disclosure comprises a water insoluble ingredient.

The term “water-insoluble” means those compounds which are either completely or partially insoluble in water.

Preferably, the water insoluble ingredient is selected from hectorite, clay, pearlescent, exfoliant and mixture thereof. According to one embodiment, the water insoluble ingredient, may be, for example, perlite.

Among clays, mention may be made of clays of the smectite family, such as laponite, of the kaolinite family, such as kaolinite, dickite or nacrite, optionally modified clays of the halloysite, dombassite, antigorite, benthierine or pyrophyllite family, montmorillonites, beidellite, vermiculites, talc, stevensite, hectorites, saponites, chlorites, sepiolite and illite.

Clays include products that are described, for example, in the publication Mineralogie des argiles [Mineralogy of Clays], S. Caillere, S. Henin, M. Rautureau, 2nd Edition 1982, Masson, which is incorporated herein by reference in its entirety. Natural clay is a sedimentary rock in large part composed of specific minerals, silicates, generally, of aluminum. Kaolin is a natural clay. The clays may also be synthetic. Clays can also be chemically modified by various compounds, such as acrylic acids, polysaccharides (for example carboxymethylcellulose) or organic cations. In some instances, the cosmetic compositions of the instant disclosure includes a clay selected from the group consisting of kaolinite, montmorillonites, saponites, laponites, hectorites (including disteardimonium hectorite), and illites.

Among pearlescent, mention may be made of ethylene glycol ester or esters. In some embodiments, the ethylene glycol esters are selected (s) from ethylene glycol esters and fatty acids, C 8-C 22, preferably C 10-C 20, preferably C 16-C 18, preferably the ethylene glycol ester is selected from glycol stearate, glycol distearate, and mixtures thereof; preferably is glycol distearate.

pH

The pH of the cleanser composition can vary but is typically about to less than 7, i.e., it is acidic. In various embodiments, the pH of the cleanser compositions is from about 3 to about 7, about 3.5 to less than 7, about 4 to about 7, about 3 to about 6.5, about 3.5 to about 6.5, about 4 to about 6.5, about 3 to about 6, about 3.5 to about 6, about 4 to about 6, about 3 to about 5.5, or about 3.5 to about 5.5.

### Optional Ingredients

In some embodiments, there may be one or more actives present in the cosmetic composition, according to the disclosure, the additive selected from, for example, humectants, such as acetamide MEA, glycols, such as glycerin and propylene glycol; alcohol; anti-microbial components, salicylic acid, alpha acid; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid; and vitamins and vitamin derivatives, such as tocopherol and ascorbic acid; and combinations thereof.

In some embodiments, there may be one or more other components present in the cosmetic composition, according to the disclosure, the other components selected from, fillers such as clays, talc, organic thickeners with for instance, anionic, cationic, nonionic, and amphoteric polymeric associative thickeners and combinations thereof; penetrants; sequestrants; fragrances; dispersants; film-forming agents; ceramides; opacifiers and combinations thereof. Although the aforementioned optional components are given as an example, it will be appreciated that other optional components compatible with cosmetic applications known in the art may be used.

In accordance with the various embodiments, the amount of actives and other components present in the composition can range from about 0 to about 50%, from about 0.5 to about 30%, from about 1.5 to about 20%, and from about 5 to about 15%, or any suitable combination, sub-combination, range, or sub-range thereof by weight, based on the weight of the composition.

In some embodiments, one or more actives, alone or in combination, can be present in the composition according to the disclosure from about 0.05 to about 50% by weight, from about 0.05% to about 2.5% by weight, from about 0.1 to about 2%, from about 0.25 to about 1.5%, and from about 0.5 to about 1.25%, or any suitable combination, sub-combination, range, or sub-range thereof by weight, based on the weight of the composition.

In some embodiments, one or more other components, such as preservatives, vitamins, preservatives, and the like, alone or in combination, can be present in the composition according to the disclosure from about 0.05 to about 50% by weight, from about 0.05% to about 25% by weight, from about 0.1 to about 10%, from about 0.25% to about 5%, and from about 0.5 to about 3.5%, or any suitable combination, sub-combination, range, or sub-range thereof by weight, based on the weight of the composition. In some exemplary embodiments, preservatives may include sodium salicylate, and vitamins may include ascorbic acid, tocopherol and combinations of these.

Thus, one or a combination of optional components may be present, by weight, based on the total weight of the composition, each one or the combination present from about 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

The above ingredients lists are only examples and not limiting.

The compositions according to the instant disclosure may be prepared according to techniques that are well known to those skilled in the art, in particular those intended for the preparation of shaving compositions.

The instant disclosure will be better understood from the examples that follow, all of which are intended for illustrative purposes only and are not meant to limit the scope of the instant disclosure in any way.

As used herein, the terms “comprising,” “having,” and “including” are used in their open, non-limiting sense.

The terms “a,” “an,” and “the” are understood to encompass the plural as well as the singular.

The expression “at least one” means one or more and thus includes individual components as well as mixtures/combinations.

The expression “one or more” as used herein includes individual components as well as mixtures/combinations.

All ranges and values disclosed herein are inclusive and combinable. For examples, any value or point described herein that falls within a range described herein can serve as a minimum or maximum value to derive a sub-range, etc.

All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc.

Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### EXAMPLES

The following Examples are intended to be non-restrictive and explanatory only. The ingredient amounts in the compositions/formulas described below are expressed in % by weight, based on the total weight of the composition.

### Example 1—Inventive Composition

TABLE 1
Inventive Example

| | | Inventive |
|---|---|---|
| | INCI US Name | Ex. 1 |
| SKIN ACTIVE | SALICYLIC ACID | 1.0-2.00 |
| INGREDIENT | | |
| SURFACTANT | SODIUM COCOYL ISETHIONATE | 0.8-1.2 |
| POLYMER | ACRYLATES COPOLYMER¹ | 0.4-0.70 |
| WATER SOLUBLE | HECTORITE | 0.06-0.20 |
| INGREDIENT | | |
| SURFACTANT | SODIUM LAUROYL | 4.1 |
| | SARCOSINATE | |
| SURFACTANT | PEG-150 PENTAERYTHRITYL | 1.30 |
| | TETRASTEARATE | |
| SURFACTANT | SODIUM METHYL COCOYL | 1.46 |
| | TAURATE | |
| SURFACTANT | COCAMIDOPROPYL | 5.00 |
| | HYDROXYSULTAINE | |
| TOTAL | | 12.85 |
| SURFACTANTS | | |
| SOLVENT | WATER | Q.S. |

| | |
|---|---|
| Other miscellaneous emulsifiers/ | ≤4 |
| surfactants, Salts, | |
| Preservatives, pH Adjusters, | |
| Fragrances, Colorants, Chelants, | |
| Extracts, Fillers, Absorbants, | |
| Additional Skin Actives, etc. | |

¹= TRADE NAME = CARBOPOL AQUA SF-1 POLYMER - MANUFACTURER = LUBRIZOL

### Example 2: Comparative Examples

TABLE 2
Comparative Examples

| | INCI US Name | Comp. Ex. 1 | Comp. Ex. 2 | Comp. Ex. 3 |
|---|---|---|---|---|
| SKIN ACTIVE | SALICYLIC ACID | 2.000 | 2.000 | 2.000 |
| INGREDIENT | | | | |
| SURFACTANT | SODIUM COCOYL ISETHIONATE | 0 | 0 | 0 |
| POLYMER | ACRYLATES COPOLYMER¹ | 0 | 0 | 0.600 |
| WATER INSOLUBLE | HECTORITE | 0.100 | 0.300 | 0.010 |
| INGREDIENT | | | | |
| OTHER SURFACTANTs | SODIUM LAUROYL | 1-13 | 1-13 | 1-13 |
| | SARCOSINATE, PEG-150 | | | |
| | PENTAERYTHRITYL | | | |
| | TETRASTEARATE, SODIUM | | | |
| | METHYL COCOYL TAURATE, | | | |
| | COCAMIDOPROPYL | | | |
| | HYDROXYSULTAINE | | | |
| TOTAL SURFACTANT | | 12.150 | 11.550 | 11.430 |
| PRESERVATIVE | CAPRYLYL GLYCOL; CALCIUM | 1.5 | 1.5 | 1.5 |
| | GLUCONATE; SODIUM | | | |
| | BENZOATE; | | | |
| | GLUCONOLACTONE | | | |
| SOLVENT | WATER | Q.S. | Q.S. | Q.S. |
| Other miscellaneous emulsifiers/surfactants, Salts, | | ≤4 | ≤4 | ≤4 |
| Preservatives, pH Adjusters, Fragrances, Colorants, | | | | |
| Chelants, Extracts, Fillers, Absorbants, Additional Skin | | | | |
| Actives, etc. | | | | |
| | | Comp. Ex. 4 | Comp. Ex 5 | Comp. Ex. 6 |
| SKIN ACTIVE | SALICYLIC ACID | 2.000 | 2.000 | 2.000 |
| INGREDIENT | | | | |
| SURFACTANT | SODIUM COCOYL | 0 | 0.500 | 1.00 |
| | ISETHIONATE | | | |
| POLYMER | ACRYLATES COPOLYMER* | 0.600 | 0.600 | 0.000 |
| WATER INSOLUBLE | HECTORITE | 0.100 | 0.100 | 0.100 |
| INGREDIENT | | | | |
| OTHER SURFACTANTS | SODIUM LAUROYL | 1-13 | 1-13 | 1-13 |
| | SARCOSINATE, PEG-150 | | | |
| | PENTAERYTHRITYL | | | |
| | TETRASTEARATE, SODIUM | | | |
| | METHYL COCOYL TAURATE, | | | |
| | COCAMIDOPROPYL | | | |
| | HYDROXYSULTAINE | | | |
| TOTAL SURFACTANT | | 11.45 | 12.53 | 12.84 |
| Preservative | CAPRYLYL GLYCOL; CALCIUM | 1.5 | 1.5 | 1.5 |
| | GLUCONATE; SODIUM | | | |
| | BENZOATE; | | | |
| | GLUCONOLACTONE | | | |
| solvent | WATER | Q.S. | Q.S. | Q.S. |

| | | | |
|---|---|---|---|
| Other miscellaneous emulsifiers/surfactants, Salts, | ≤4 | ≤4 | ≤4 |
| Preservatives, pH Adjusters, Fragrances, Colorants, | | | |
| Chelants, Extracts, Fillers, Absorbants, Additional | | | |
| Skin Actives, etc. | | | |

¹= TRADE NAME = CARBOPOL AQUA SF-1 POLYMER - MANUFACTURER = LUBRIZOL

### Example 3: Demonstration of Stability

Cleansers with high levels of salicylic acid are difficult to stabilize when attempting to suspend water insoluble materials and maintain viscosity. The instant disclosure described that the association of Sodium Cocoyl Isethionate with the acrylate copolymer allowed for the water insoluble material to stay in suspension while maintaining the high level of salicylic acid within a pH range of 5-7

To evaluate the stabilization of the cleansers, experiments were conducted on several formulas with different levels of Sodium Cocoyl Isethionate as well as different level of Acrylate copolymer and different level of Hectorite.

In all the formulas, the total amount of ingredients was maintained in order to be able to compare the inventive and comparative formulas.

The results are presented in Table 3 below.

TABLE 3

| | Inv. Ex. 1 | Comp. Ex. 1 | Comp. Ex. 2 | Comp. Ex. 3 |
|---|---|---|---|---|
| SODIUM COCOYL ISETHIONATE | 1 | 0 | 0 | 0 |
| ACRYLATE COPOLYMER * | 0.6 | 0 | 0 | 0.600 |
| HECTORITE (Water insoluble | 0.1 | 0.100 | 0.300 | 0.010 |
| ingredient) | | | | |
| TOTAL SURFACTANT | 12.85 | 12.15 | 11.55 | 11.43 |
| APPEARANCE | No | aesthetic | 55 C. sample | Separation |
| | separation | phase | separated | seen at 55 C. |
| | | separation | | (1 week) |
| | | and formula | | |
| | | thins out at | | |
| | | 45 C. on | | |
| | | stability. | | |
| STABILITY RESULSTS | Stable | not stable | not stable | Add acrylate, |
| | formula (3 | | | no SCI, |
| | months | | | not stable |
| | stability) | | | |

| | Comp. Ex. 4 | Comp. Ex. 5 | Comp. Ex. 6 |
|---|---|---|---|
| SALICYLIC ACID | 2.000 | 2.000 | 2.000 |
| SODIUM COCOYL ISETHIONATE | 0 | 0.500 | 1.000 |
| ACRYLATE COPOLYMER * | 0.600 | 0.600 | 0.000 |
| HECTORITE (Water insoluble | 0.100 | 0.100 | 0.100 |
| ingredient) | | | |
| TOTAL SURFACTANT | 11.45 | 12.53 | 12.84 |
| APPEARANCE | Separation | Separation/ | Separation |
| | seen at 55 c. | precipitation | seen at 55 C. |
| | | seen at 55 C. | |
| STABILITY RESULTS | increase | add SCI with | No SCI, |
| | acrylate, no | acrylate not | unstable |
| | SCI, not | stable | |
| | stable | | |

* = Trade Name = CARBOPOL AQUA SF-1 POLYMER - Manufacturer = LUBRIZOL

### Results Interpretation

The comparison between the Inventive Example 1 and the Comparative Examples allowed to show that the presence of the sodium cocoyl isethionate, the acrylate copolymer and the hectorite need to be within a certain ranges in order to have stable compositions.

Several cases were studied and discussed below.

### Inventive Example 1/Comparative Example 1

It was shown that Comparative Example 1 that didn't contain sodium cocoyl isethionate nor acrylate copolymer but contained the same amount of hectorite lead to a formula that was thin, showed separation and was unstable at 45 C.

### Inventive Example 1/Comparative Example 2

It was shown that Comparative Example 2 that didn't contain sodium cocoyl isethionate nor acrylate copolymer but contained a higher amount of hectorite lead to a formula that showed separation and was unstable at 55 C.

### Inventive Example 1/Comparative Example 3

It was shown that Comparative Example 3 that didn't contain sodium cocoyl isethionate but did contain the same amount of acrylate copolymer as Inventive Example 1 and a lower amount of hectorite as Inventive Example 1 lead to a formula that showed separation and was unstable at 55 C.

### Inventive Example 1/Comparative Example 4

It was shown that Comparative Example 4 that didn't contain sodium cocoyl isethionate but did contain the same amount of acrylate copolymer and hectorite as Inventive Example 1 lead to a formula that showed separation and was unstable at 55 C.

### Inventive Example 1/Comparative Example 5

It was shown that Comparative Example 5 that contain the same amount of sodium cocoyl isethionate as well as hectorite as Inventive Example 1 but didn't contain acrylate copolymer lead to a formula that showed separation and was unstable at 55 C.

### Inventive Example 1/Comparative Example 6

It was shown that Comparative Example 6 that contain a lower amount of sodium cocoyl isethionate but did contain the same amount of acrylate copolomer and hectorite as Inventive Example 1 lead to a formula that showed separation and was unstable at 55 C.

While the disclosure has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A cleanser composition comprising:
a. salicylic acid;
b. a first surfactant sodium cocoyl isethionate;
c. at least one acrylate copolymer;
d. a water insoluble ingredient; and
wherein the cleanser is stable and the water insoluble ingredient stays in suspension; and
wherein all weight percentages are based on a total weight of the cleanser composition.

2. The cleanser composition of claim 1, wherein salicylic acid is present from about 0.5 to 2 wt. % based on the total weight of the cleanser composition.

3. The cleanser composition of claim 1, wherein the sodium cocoyl isethionate is present from about 0.5 to 10 wt. % based on the total weight of the cleanser composition.

4. The cleanser composition of claim 1, wherein the at least one acrylate copolymer is selected from crosslinked copolymers containing at least one monomer selected from methacrylic acid; acrylic acid; amino acrylic acid; an acrylic acid ester of a C8-C30 alkyl or alkenyl, which may be substituted; a methacrylic acid ester of a C8-C30 alkyl or alkenyl, which may be substituted; a C1-C4 alkyl acrylate and a C1-C4 methacrylate, which may be substituted.

5. The cleanser composition of claim 4, wherein the at least one the acrylates copolymer is selected from crosslinked copolymers comprising at least one methacrylic acid unit and at least one C1-C4 alkyl acrylate unit.

6. The cleanser composition of claim 1, wherein the at least one acrylate copolymer is present from about 0.1 to 4 wt. % based on the total weight of the cleanser composition.

7. The cleanser composition of claim 1, wherein the at least one acrylate copolymer is present from about 0.2 to 3.5 wt. % based on the total weight of the cleanser composition.

8. The cleanser composition of claim 1, wherein the water insoluble ingredient is present from about 0.05 to about 10 wt. % based on the total weight of the cleanser composition.

9. The cleanser composition of claim 1, wherein the water insoluble ingredient is selected from the group consisting of hectorite, clay, pearlescent, exfoliant and mixture thereof.

10. The cleanser composition of claim 1, further comprising a second or more surfactants.

11. The cleanser composition of claim 10, wherein the second or more surfactants is selected from the group consisting of anionic, ionic, amphoteric surfactants and mixture thereof.

12. The cleanser composition of claim 10, wherein the second or more surfactants are present from about 0.5 to about 20 wt. % based on the total weight of the cleanser composition.

13. The cleanser composition of claim 1 having a pH of about 5 to about 7.

14. A cleanser composition comprising:
a. from about 0.5 to 2 wt. % of salicylic acid;
b. from about 0.5 to 10 wt. % of a first surfactant sodium cocoyl isethionate;
c. from about 0.1 to 4 wt. % of at least one acrylate copolymer;
d. from about 0.05 to about 10 wt. % of a water insoluble ingredient; and
wherein the cleanser is stable and the water insoluble ingredient stays in suspension; and
wherein all weight percentages are based on a total weight of the cleanser composition.

15. The cleanser composition of any one of preceding claims, further comprising one or more adjuvants commonly used in the cosmetic field selected from the group consisting of cosmetic or dermatological active principles, fragrances, preservatives, sequestering agents, pigments, pearlescent agents, inorganic or organic fillers, powder or polyethylene powder, soluble dyes, sunscreen agents and their mixtures.

16. A method for cleansing the keratin materials, preferably the skin, more preferably the face, comprising a step of applying the cleanser composition of claim 1 to said keratin materials.

17. Use of the cleanser composition of any one of preceding claims in cleansing the keratin material, preferably the skin, more preferably the face.
